(19) United States
(12) Patent Application Publication
(10) Pub. No.: US 2019/0160865 A1
(43) Pub. Date: May 30, 2019

(54) MEDICAL DEVICE WITH AN OMNI-WHEEL AND AN OMNI-WHEEL ARRANGEMENT

(71) Applicant: KONINKLIJKE PHILIPS N.V., EINDHOVEN (NL)

(72) Inventors: Ravindra BHAT, EINDHOVEN (NL); Shailesh Kumar AGRAHARI, PUNE (IN)

(21) Appl. No.: 16/323,126

(22) PCT Filed: Aug. 4, 2017
(86) PCT No.: PCT/EP2017/069873

§ 371 Date: Feb. 4, 2019

(30) Foreign Application Priority Data
Aug. 5, 2016 (EP) 16183013.8

(51) Int. Cl. B60B 19/00; B60B 19/12; B60K 7/00; B60T 1/02

(52) U.S. Cl. CPC B60B 19/003; B60B 19/12; B60L 7/00; B60T 1/02; B60K 7/0007

(57) ABSTRACT

The present invention relates to an omni wheel (312′, 312″, 312′″) and an omni wheel arrangement allowing for easy movement of a medical device by using braking devices (307a, 307b, 307c, 307d).

### FIELD OF THE INVENTION

The present invention relates to medical devices. In particular, the present invention relates, to a mobile surgery system comprising at least one omni wheel, to an omni wheel, to an omni wheel arrangement, to a method for controlling an omni wheel, to a program element and/or to a computer-readable medium.

### BACKGROUND OF THE INVENTION

A mobile surgery system and in particular a mobile surgery imaging system including a C-arm, is a device with an X-ray source and an X-ray detector mounted on a C-shaped carriage system. X-ray systems are particularly used for diagnostic purposes, e.g. for acquiring X-ray images of an object to be examined.

For acquiring X-ray images, an X-ray system regularly comprises an X-ray generating device and an X-ray detector. The X-ray generating device and the X-ray detectors are arranged on opposite sides of the object to be examined and are operatively coupled for the acquisition of X-ray images. X-ray radiation is emitted by the X-ray generating device towards the X-ray detector. An object to be examined is situated between the X-ray detector and the X-ray generating device and is thus penetrated by X-ray radiation. Detector elements of the X-ray detector acquire information in accordance with the spatially attenuated X-ray radiation, which information is subsequently used for the generation of an X-ray image of the object.

Regularly, both the X-ray generating device and the X-ray detector are heavy and rigidly built elements having a substantial weight. Thus, for moving an X-ray system on a surface, e.g. on the floor of an operating room, a substantial force has to be applied to the X-ray system for desired placement and positioning. In particular, precise positioning with regard to the object to be examined may be required.

Document WO 2011/030255 A1 relates to a bidirectional movement assembly.

### SUMMARY OF THE INVENTION

There may be a need for a more efficient movement of a mobile surgery system.

The object of the present invention is solved by the subject-matter of the independent claims, wherein further embodiments are incorporated in the dependent claims.

According to an aspect of the present invention, a mobile surgery system is provided. The mobile surgery system comprises at least one omni-wheel for moving the mobile surgery system, e.g. a mobile imaging surgery system including a C-arm, wherein the omni-wheel comprises at least one wheel device having a wheel body, a wheel shaft arranged in the center of the wheel body, at least one roller device, at least one of a wheel braking device and/or a roller braking device. The at least one roller device has a roller shaft and a roller body, wherein the at least one roller device is arranged on the wheel body such that at least a part of the circumferential surface of the roller device forms at least a part or a portion of a circumferential surface of the wheel body. The wheel braking device, if available, is adapted for blocking a movement of the wheel body around the wheel shaft or about a corresponding axis. Accordingly, if a roller braking device is available, the roller braking device is adapted for blocking a movement of the roller body around the roller shaft or about a corresponding axis.

In an example an omni wheel is provided, where the omni wheel comprises at least a wheel device having a wheel body, a wheel shaft arranged in the center of the wheel body, at least one roller device, and at least one of a wheel braking device and/or a roller braking device. A wheel braking device is adapted for stopping, blocking and/or locking a rotation of the wheel body around the wheel shaft. A roller braking device is adapted for stopping, blocking and/or locking a rotation of the roller device. The wheel braking device and roller braking device may be activated independent from each other.

For example, in an embodiment wherein more than one omni wheel is used, an omni wheel may either have only a wheel braking device for blocking the wheel or a roller braking device for blocking at least one of the rollers. In an example the omni wheel may also have both, a wheel braking device and a roller braking device. This may be the case when a single omni wheel is used, or alternatively when more than one omni wheel is used, as well.

The at least one roller device has a roller shaft and a roller body. In one example the at least one roller device is arranged in a constant distance from the wheel shaft such that the roller body forms at least a part of a wheel circumference of the wheel body for contacting the surface. In another example the at least one roller device is arranged in a variable distance from the wheel shaft. The shaft of the roller may be mounted on a spring or on another elastic element in order to be able to compensate irregularities of a surface of a floor on which the omni-wheel may rolling. The wheel braking device is adapted for blocking a movement of the wheel body around the wheel shaft or about the wheel shaft. The roller braking device is adapted for blocking a movement of the roller body around the roller shaft or about the roller shaft. In an example where a wheel device comprises a roller device, the shaft of the wheel device may substantially be oriented perpendicular to the shaft of the roller device. In other words, the wheel device may support a movement perpendicular to the movement supported by the roller device.

According to an aspect of the invention an omni-wheel is described which comprises a wheel device having a wheel body, a wheel shaft, a wheel axis or a wheel axle arranged in the center of the wheel body, at least one roller device, at least one of a wheel braking device and/or a roller braking device. The at least one roller device has a roller shaft and a roller body wherein the at least one roller device is arranged on the wheel body such that at least a part of the circumferential surface of the roller device forms at least a part of a circumferential surface of the wheel body. The wheel braking device is adapted for blocking a movement of the wheel body around the wheel shaft or about the wheel shaft. Furthermore, if available, the roller braking device is adapted for blocking a movement of the roller body around the roller shaft or about the roller shaft.

According to another aspect of the invention an omni-wheel arrangement is provided, comprising a first omni-wheel and a second omni-wheel as well as a mounting plate. The first omni-wheel and the second omni-wheel are mounted on the same side of the mounting plate. The omni-wheel arrangement may be used as a basis for a mobile surgery system.

In an example an omni wheel arrangement is provided comprising a first omni wheel and a mounting plate. The first omni wheel is mounted on the mounting plate. In another example, a medical device or a mobile surgery imaging system including a C-arm comprises at least one of an omni wheel and an omni wheel arrangement.

According to an aspect of the invention a method for operating or controlling an omni-wheel is presented, the method comprising determining a direction of desired movement. The desired movement may be a user input provided via an input device. If the direction of desired movement is parallel to the wheel shaft the wheel braking device is activated and in this way the corresponding wheel is blocked, while the roller braking device is not activated enabling the desired movement by means of the roller device. If the direction of desired movement is perpendicular to the wheel shaft the roller braking device is activated and the shaft of the roller device is blocked, while the wheel braking device is not activated enabling the desired movement by means of the wheel body rotating around the wheel shaft. If however, no movement is desired both, the wheel braking device and the roller braking device are activated.

According to another aspect of the present invention, a method for operating or controlling a mobile surgery system is provided comprising determining a direction of a desired movement. If the direction of desired movement is parallel to the wheel shaft of the at least one omni wheel the wheel braking device of the wheel of the omni wheel is activated and the wheel braking device of the further omni wheel perpendicular to the at least one omni wheel is unlocked. If the determined direction of desired movement is perpendicular to the wheel shaft of the at least one omni wheel or of the first wheel the roller braking device of the at least one omni wheel activated and/or the wheel braking device of the further omni wheel or of the second wheel is activated. If no movement is desired the wheel braking device of at least one omni wheel and/or the wheel braking device of the further omni wheel are activated.

In an example a further method for operating an omni wheel may be provided. The method comprises determining a direction of a desired movement. If the direction of desired movement is parallel to the wheel shaft, the method comprises activating the wheel braking device. By using a braking device, the wheel can be locked. If the direction of the desired movement is perpendicular to the wheel shaft, the roller braking device is activated. Activating the roller braking device results in blocking at least one of the rollers.

If no movement is desired, both, the wheel braking device and the roller braking device are activated in order to block the wheel as well as the roller.

In another example a method for operating or controlling an omni wheel may comprise determining a direction of desired movement. The desired or intended direction may be provided by a user via a user interface such as a button, a joystick or a smart handle, which is adapted to recognize a desired movement by the orientation of the handle, by the strength and/or by the direction of the applied force. Dependent on the detected direction the action with regards to the brakes is executed. If the direction of the desired movement is parallel to the first wheel shaft of the first wheel, e.g. if a wig-wag movement is to be made, the method may comprise activating the wheel braking device of that first wheel and unlocking the wheel braking device of the wheel perpendicular to the first wheel, if such a braking device is available. If a wheel braking device is not available releasing and/or activating the wheel braking device is not necessary. If a roller braking device is not available releasing and/or activating the roller braking device is not necessary.

Furthermore, the method may comprise activating the roller braking device of the first wheel and/or activating the wheel braking device of the second wheel, in case the direction of the desired movement is perpendicular to the first wheel shaft, e.g. if a longitudinal movement is to be made. In an example, where no roller braking device is available only the wheel braking device of the second wheel may be activated.

If no movement is desired, the wheel braking device of the first wheel and of the second wheel may simultaneously be activated.

The direction of the movement can be selected by a user interface connected to the wheel braking device and/or connected to the roller braking device. A user interface may be a button or a sensor such as a force sensing handle or a smart handle which translates the intended direction by switching on and off the respective brakes.

According to another aspect of the present invention, a program element for operating or controlling an omni wheel and/or an omni wheel arrangement is provided.

According to yet another aspect of the present invention, a computer-readable medium, such as a CD-ROM, comprising a program code is provided, which, when being executed by a processor, is adapted to carry out at least one of the method of operating an omni wheel and/or the method of operating an omni wheel arrangement. In particular, the method, the program element and the computer-readable medium, respectively are adapted to operate a braking device of a wheel device and/or of a roller device.

A program element may also be presented over a network like the World Wide Web and can be downloaded into the working memory of a data processor from such a network.

According to a further exemplary embodiment of the present invention, a medium for making a computer program element available for downloading is provided, which computer program element is arranged to perform a method according to one of the previously described embodiments of the invention.

A mobile surgery system and in particular a mobile surgery imaging system including a C-arm is a device with an X-ray source and an X-ray detector mounted on a C-shaped structure and is commonly used for orthopaedic surgeries, pain management, urology, vascular clinical segments, etc. Operation theatres may be very crowded. Using an omni wheel on the C-arm system may allow for reducing the space which is used by the C-arm since certain movements which are desired to be fulfilled by a mobile surgery system and/or a C-arm system are made by an omni wheel mounted on the basis of the C-arm system. The omni wheel may be used instead of a complicated and bulky mechanism for moving the C-arm. Therefore, more space may be available for the personnel involved in the surgery. The mobile surgery system has various fine positioning movements such as angulation, propeller, wig-wag, height positioning in order to position the human anatomy of interest within the field of view of the ‘image chain’. An ‘image chain’ may be a term used to summarize an X-ray source, a detector, a frame grabber, and a device for processing and for displaying of the image. For long bone surgeries, e.g. femoral fractures, or spine surgeries where fine positioning movements are insufficient to cover the entire anatomy of interest, the mobile surgery system is moved within the operating room (OR) by means of a parallel movement using base wheels. The system may have to satisfy regulatory clauses related to overbalance and unwanted lateral movements on 5° and 10° ramps. These regulatory clauses are currently often fulfilled by using counterbalancing the location of the center of gravity in the worst possible orientation. To be on the safe side, balance weights are added in locations farthest away from the C-arc and may make the system bulky and heavy and hence difficult to maneuver.

The wheel location is based on the locus of centers of gravity in different orientations of the system. CGs (center of gravity) are to be covered in order to balance the system by a wheel base footprint that needs to be broad enough to ensure a balanced weight contribution in all possible situations.

Using the omni wheel instead of a bulky mechanism for the longitudinal and wig-wag movement of the C-arc, may allow for reducing the footprint of CG distribution and the footprint of the wheel base can be minimized resulting in a compact system design. By using the omni wheel the wig-wag mechanism and longitudinal direction motion can be reduced and the CGs are not widely spread.

The omni wheel of the present invention uses braking devices within the omni wheel. Using a braking device allows for a manual movement of the mobile surgery system and does not need a motor-assisted system for all movements. The motor-assisted system may increase the cost of the system due to the use of multiple motors and may also increase the bulkiness of the system. However, motors can be used either as a braking device or in addition to a braking device in order to support the manual movement for particularly heavy devices. Also the braking function of a motor may be used for locking the wheel. In specific applications a motor may also be provided for braking the rollers. In an example it may be possible to provide a low cost manual system by using electromagnetic brakes. In one example electromagnetic brakes are installed for locking a movement of a rotating device. In a further example electromagnetic brakes are only arranged on the wheel devices in order to lock and unlock a movement of a wheel. An electromagnetic brake may be mounted on a wheel shaft of the wheel whose rotation is to be controlled.

A brake can be replaced with a motor which can work as a brake when the motor is brought into a stop state. A motor, e.g. an electromotor, may also rotate to assist the movement for an easy handling of the medical device or to support a movement of a heavy device.

Consequently, a system such as a medical device using an omni wheel can reduce the space consumed by such a system. This reduction and prevention of a bulky device can help saving precious real estate in hospital environment and reduce the positioning issues in crowded operation rooms. Using omni wheels may help to reduce the footprint of the C-stand and may allow for a good placement in crowded OT (operation theatres).

Due to the compact design of the mobile surgery system when using omni wheels with respective braking devices, the weight of the mobile surgery system may be low and such a system may be easily to designed and/or dimensioned. The weight may be reduced because balancing masses to protect against over-balancing may not be necessary with a compact design of the medical system and only a low movement force may be necessary to move the medical system, in particular when moving the system in a longitudinal and/or in a wig-wag movement.

The omni wheels thus allow for a low weight medical system which may easily be handled manually by an operator or user of the system. An omni wheel may also help to increase the preciseness of a movement since the omni wheel can very precisely determine the direction of movement preferably by means of independently activating the wheel braking device and the roller braking device. Thus, the omni wheel using brakes may for example provide well-defined wig-wag and longitudinal movements for a C-arm imaging systems. Effects of skewed application of force, which can render maneuvering the C-arm more difficult, can thereby be reduced.

According to an aspect of the invention the at least one roller device is movable within the wheel device. In an example the roller device may be mounted using a spring or another elastic element that may allow the roller device to compensate irregularities of a surface and to ensure a good contact between the circumferential surface of the roller device and the wheel device, respectively and the surface on which the mobile surgery system is moved.

According to another aspect of the present invention, the diameter of the roller device is smaller than the diameter of the wheel device. A reduced diameter may make it possible that the roller device as a complete device is moving together with the wheel body around the wheel shaft. The surface of the roller device can also form the surface of the wheel device.

If the roller body is made of a material having a high friction number a wheel with a resulting high friction may be provided. For example, the roller body may be made of rubber whereas the wheel body may be made of another material.

According to another aspect of the present invention, at least one of a wheel braking device and a roller braking device comprises at least one of a braking device, a motor and/or a shaft locking device. A braking device may be a plate that is pressed against the roller. In an example an electromagnetic brake may be used that is mounted on a shaft of the wheel device that is to be controlled. A motor that is brought into a stop state may be an alternative example for a braking device controlling the motion of a wheel device.

Dependent on the design of the omni wheel, different braking methods may be applicable with regard to size and strength of the braking force.

In an example the mobile surgery system and in particular the omni wheel arrangement comprise a first omni wheel and a second omni wheel. The second omni wheel may be mounted on the same side of the mounting plate as the first omni wheel. In a further example the first omni wheel and the second omni wheel are mounted on the mounting plate or on the basis of the mobile surgery system in such a way that the shaft of the wheel device of the first omni wheel lies on the same virtual axis as the shaft of the wheel device of the second omni wheel.

Such a configuration may be named as double wheel configuration and may be used for heavy weights of the medical device.

According to another aspect of the present invention, the mobile surgery system may further comprise a further omni-wheel or a second omni-wheel and a mounting plate wherein the at least one omni-wheel or the first omni wheel and the further omni-wheel are mounted on the same side of the mounting plate. Thus in an operational status, both the at least one wheel and the further omni wheel may be positioned as to bee between the base plate and a floor.

In an example, the first omni wheel and the second omni wheel are mounted on the mounting plate in such a way that the shaft of the wheel device of the first omni wheel lies on a virtual axis perpendicular to the virtual axis of the shaft of the wheel device of the second omni wheel. In other words, the shafts of the first omni wheel are in a T-shape configuration oriented to another.

According to another aspect of the present invention at least one omni wheel and the further omni-wheel are mounted on the mounting plate such that the shaft of the wheel-device of the at least one omni-wheel lies on the same virtual axis as the shaft of the wheel-device of the further omni-wheel. Such a double wheel construction may help distributing a load to at least two wheels.

According to another aspect of the present invention the at least one omni wheel and the further omni-wheel are mounted on the mounting plate such that the shaft of the wheel-device of the at least one omni-wheel lies on a virtual axis perpendicular to the virtual axis of the shaft of the wheel-device of the further omni-wheel.

Such a T-shape configuration may support movements into different directions. In particular movements in directions perpendicular to another may be supported.

According to yet another aspect of the present invention, the first omni wheel and the second omni wheel are mounted in a common housing.

According to another aspect of the present invention, the first omni wheel and the second omni wheel are mounted on a pivot.

Mounting both wheels on a pivot may allow for an easy longitudinal movement in particular when crossing obstacles like small steps or cables.

### BRIEF DESCRIPTION OF THE DRAWINGS

Exemplary embodiments of the invention will be described in the following with reference to the following drawings:

FIG. 1 shows a mobile X-ray system with a front castor wheel for a better understanding of the present invention.

FIG. 2 shows a mobile surgery system according to an exemplary embodiment of the present invention.

FIG. 3 shows a cross sectional side view of an omni wheel arrangement according to an exemplary embodiment of the present invention.

FIG. 4 shows a bottom view of an omni wheel arrangement allowing for a multidirectional movement according to an exemplary embodiment of the present invention.

FIG. 5 shows the omni wheel configuration of FIG. 4 with locked rollers according to an exemplary embodiment of the present invention.

FIG. 6 shows the omni wheel arrangement of FIG. 4 with a locked wheel movement according to an exemplary embodiment of the present invention.

FIG. 7 shows the omni wheel arrangement of FIG. 4 where wheels and rollers of the omni wheel are blocked according to an exemplary embodiment of the present invention.

FIG. 8 shows an omni wheel arrangement with two parallel omni wheels mounted in a front part of a platform according to an exemplary embodiment of the present invention.

FIG. 9 shows a double omni wheel sideways rolling configuration according to an exemplary embodiment of the present invention.

FIG. 10 shows an omni wheel arrangement using a combination of forward and sideways oriented omni wheels according to an exemplary embodiment of the present invention.

FIG. 11 shows a pivot mounting configuration of two omni wheels according to an exemplary embodiment of the present invention.

FIG. 12 shows a bottom view of a perpendicular omni wheel arrangement according to an exemplary embodiment of the present invention.

FIG. 13 is a front view of omni wheel arrangement of FIG. 12 according to an exemplary embodiment of the present invention.

FIG. 14 shows a side view of omni wheel arrangement of FIG. 12 according to an exemplary embodiment of the present invention.

FIG. 15 shows different views of a support plate with an omni wheel arrangement according to an exemplary embodiment of the present invention.

### DETAILED DESCRIPTION OF EMBODIMENTS

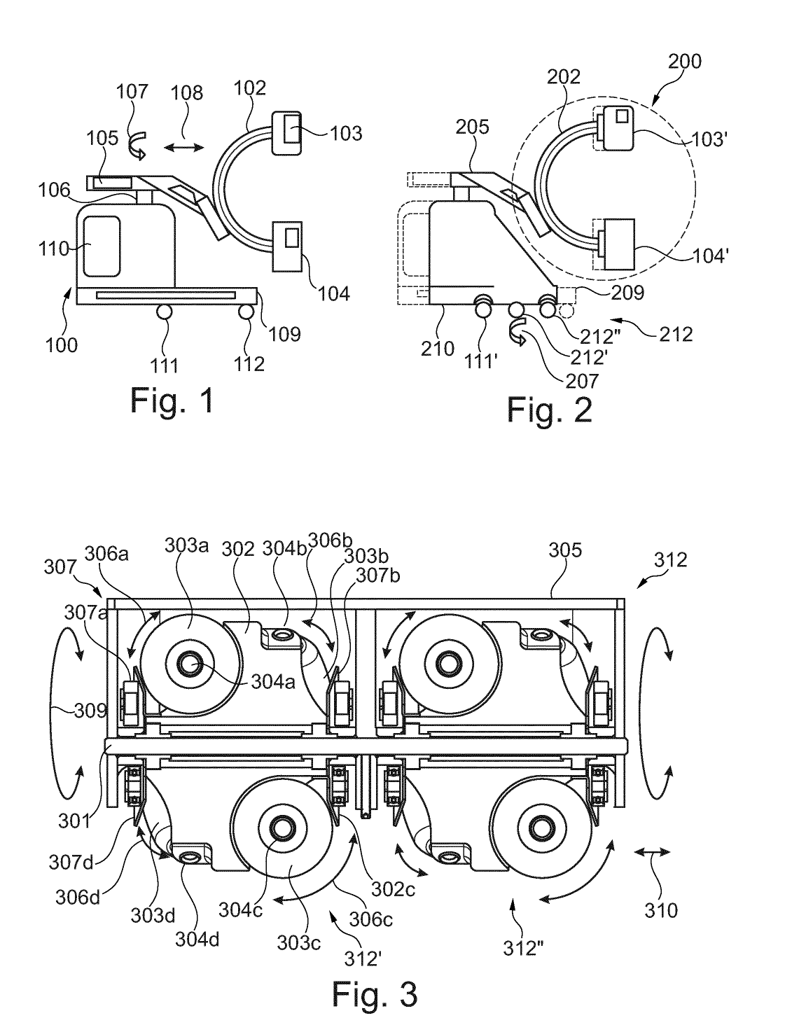

FIG. 1 shows a mobile X-ray system with a front castor wheel 112 for a better understanding of the present invention. The X-ray system 100 shows the C-arm 102 with the X-ray source 103 and the X-ray detector 104. The C-arm 102 is mounted on a support arm 105 which has a joint 106 to allow wig-wag movements 107 and longitudinal movements 108. In order to support the wig-wag movements and longitudinal movements 108, a large base plate 109 is used in order to provide a large footprint for different centers of gravity that may vary in a wide range in order to support the longitudinal and/or wig-wag movements of support arm 105. The large amount of counterweights which are installed to meet regulatory requirements leads to a bulky system body 110. The counterweights are necessary to put the center of gravity in the middle of back wheels 111 and the front castor wheel 112.

FIG. 2 shows a medical device 200, e.g. a mobile surgery imaging system 200 including a C-arm, according to an exemplary embodiment of the present invention. The mobile surgery system 200 has a system body 210 and a support arm 205 which holds the C-arm 202. Mounted on this C-arm are an X-ray source 103′ and the X-ray detector 104′. They are mounted in opposite directions facing another. In addition to the back wheels 111′, a pair of omni wheels 212′, 212″ is used. As indicated by arrow 207, the omni wheels 212, 212′, 212″ can be used to support a wig-wag movement and a longitudinal movement of the medical system 200. The use of omni wheels 212 allows for a precise movement in the longitudinal direction. Also when a wig-wag movement is performed precise movement is possible. As indicated by the dashed lines, the size of the whole system body 210 can be reduced to a compact design, compared to the extension of the system 100 using support arm 105 for making wig-wag movements. Instead of a front castor wheel 112, the combination of omni wheels 212, 212′, 212″ with the integrated brake mechanism which are mounted on the wheel basis 209 allows for more precise movement. The use of the omni wheel 212 for a variety of movements allows for implementing a wig-wag mechanism and longitudinal motion mechanism with low complexity. Consequently, the volume of the system is small. The small system reduces the footprint of the whole system and makes the system less bulky. Since the small shape of the system 200 results in a minimal amount of counterweights, the system 200 may be built in a lightweight manner and therefore may be easily moved.

The mobile X-ray system 200 with the omni wheel 212 or a corresponding omni wheel arrangement helps to precisely position the X-ray C-arm 202. The omni wheels 212, 212′, 212″ can deliver the longitudinal and wig-wag movement and do not need a complex system for supporting these movements in the support arm 105. Thus, a light and compact X-ray system 200 can be provided. If the omni wheels 212 are used precise movements in predefined directions is possible. The high precision of movements may allow for a good repetition and/or recall of movements of the mobile surgery system. E.g. may only show low jerks when executing a back and forth motion since even for 180° movements substantially no alignment of the move direction is necessary. Furthermore, the omni wheels 212, 212′, 212″ can separate two motions and/or directions of motions. The separately selectable motion in different directions may contribute to precise motion, e.g. longitudinal or wig-wag motion. In other words, separating a motion in substantially two or less motions may help to provide a high precision for positioning. The omni wheel allows for controlling each motion separately in a single direction.

Consequently, the omni wheels 212 may allow for a precise movement of the mobile surgery system. Omni wheels are capable of conducting motions in all directions. These motions may be assembled by motions into predefined directions of the omni wheel. Using the brakes inside the omni wheels allows for individually blocking the wheels' motion in the primary two directions. The braking devices thus may individually lock each of the two orthogonal directions as required. A separation of two motions may be possible, e.g. wig-wag and longitudinal.

FIG. 3 shows a cross sectional side view of an omni wheel arrangement according to an exemplary embodiment of the present invention. The omni wheel arrangement 312 comprises two individual omni wheels 312′, 312″ which can be operated and controlled separately from another. In the configuration of omni wheel arrangement 312, both omni wheels 312′, 312″are mounted in the same orientation. Since both omni wheels 312′, 312″ are operated separately and independently from another, only omni wheel 312′ is described in the following. The same description also applies for the other omni wheel 312′ of the omni wheel arrangement 312.

Omni wheel 312′ shows the wheel shaft 301 or wheel axle 301. Wheel shaft 301 and wheel body 302 form a corresponding wheel device. The wheel shaft 301 defines a longitudinal axis. The wheel body carries the rollers 303a, 303b, 303c, 303d. Rollers 303 have a roller body and are each mounted on roller shafts 304a, 304b, 304c, 304d.

The lower roller 303c can be in contact with the surface from a floor, for example a floor of an operation theatre, which is not shown in FIG. 3. The upper roller 303a, 303b are located inside the housing 305. Both omni wheels 312′, 312″ are placed within the housing 305. In order to align the circumferential surface of the wheel 321 with the circumferential surface of the rollers 303a, 303b, 303c, 303d elastic mounting devices may be used when roller shaft 304a, 304b, 304c, 304d are mounted inside wheel body 302.

A possible movement of rollers 303a, 303b, 303c, 303d about roller schaft 304a, 304b, 304c, 304d is indicated by arrows 306a, 306b, 306c, 306d. In order to block the movement of the roller 303a, 306b, 306c, 306d a braking device 307, 307a, 307b, 307c, 307d can lock the corresponding roller. The braking devices 307 comprise plates which in a blocking position can be pressed onto the rollers and in particular to the circumferential surface of a roller in order to stop a movement of the rollers around its roller shaft 304a, 304b, 304c, 304d. FIG. 3 shows a configuration, where all the braking devices 307 are released and the rollers 303a, 303b, 303c, 303d can turn around the shafts 304a, 304b, 304c, 304d.

Also wheel shaft 301 can have a braking device in order to block a wheel turning into direction indicated by arrow 309. Assumed that omni wheel 312, 312′, 312″ is mounted in such a way that a perpendicular movement to the shaft 301 and parallel to the shaft 304a is a longitudinal movement, it can be seen that the rollers 303c support a sideways movement or a wig-wag movement 310 if the omni wheels 312′, 312″ is placed on a surface. Also shaft 301 and each individual omni wheel 312′, 312″ can have a braking device for locking a wheel movement in the direction of arrow 309. An electromagnetic brake may be used as a braking device. An electromagnetic brake may comprise a rotatable inner part to be mounted on the rotating shaft of a wheel or roller and an outer part that is to be fixed on a mounting support, e.g. a housing. The electromagnetic brake is able to control the movement of the inner part compared to the outer part and thus control the movement and/or turning of the shaft 301. A spring inside the electromagnetic brake may hold the rotating inner part with friction and when the magnet is energized the shaft is free to rotate. Other designs of a brake device may also be applicable.

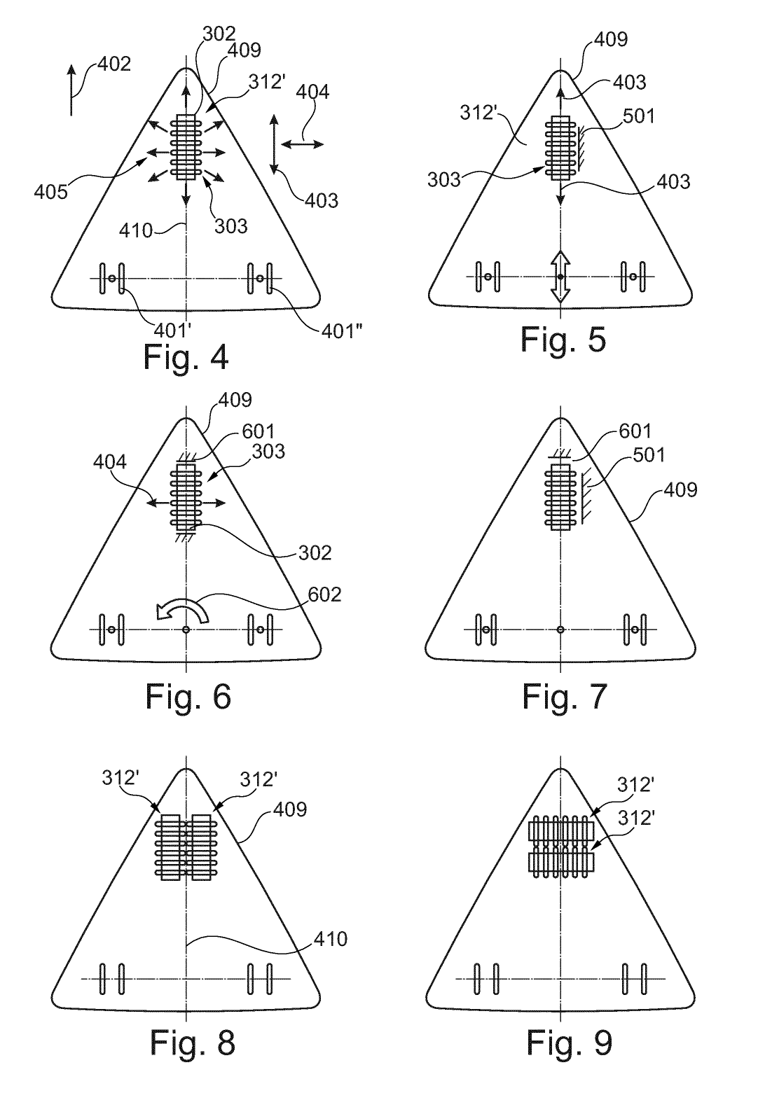

FIG. 4 shows a bottom view of an omni wheel arrangement allowing for a multidirectional movement according to an exemplary embodiment of the present invention. In FIG. 4 a base plate 409 is used having a substantially triangular shape. FIG. 4 also shows the balancing wheels 401′, 401″ which are used for stabilizing the medical system. The balancing wheels 401′, 401″ may be implemented as conventional unidirectional wheels. Omni wheel 312′ is mounted in the middle of the balancing wheels 401′, 401″ in the direction of a longitudinal forward direction 402. The omni wheel 312′ is positioned close to a pointed corner of the base plate 409. The pointed corner may indicate the forward direction 402. Omni wheel 312′ is mounted on a longitudinal axis 410 or symmetry axis 410 of the base plate 409. The longitudinal axis is parallel to a longitudinal movement. Omni wheel 312′ comprises the wheel body 302 and a plurality of rollers 303. As can be seen from FIG. 4 a movement in a plurality of directions 405 is possible. By controlling a movement of wheel 302 and/or rollers 303 e.g. by locking or unlocking a certain direction, individual directions can be separated and a precise movement may be generated. Rollers 303a, 303b, 303c, 303d can be used for transmitting force to a surface for both the movement in the wheels' direction and the movement in the rollers' direction.

The wheel body 302 is directed in the longitudinal direction 410, 403 so that a longitudinal movement involves a wheel movement instead or in addition to a roller movement. In the schematic view of FIG. 4, the rollers 303 are drawn as a plurality of parallel rollers 303 directed into the side direction 404. Any other configuration, however, is possible. The omni wheel 312′ at the front position of plate 409 separates the longitudinal direction 403 and a side direction 404 and thus allows a precise movement in any direction. Jacks when changing a direction may be prevented. The possible omnidirectional movement is indicated by the different directions 405.

FIG. 5 shows the omni wheel configuration of FIG. 4 with locked rollers according to an exemplary embodiment of the present invention. A pure longitudinal motion of the mobile surgery system supported on the platform 409 may be achieved by blocking the rollers 303 with the brakes as indicated with symbol 501 . Thus, FIG. 5 shows a locked status of the rollers 303. The roller braking device can be used to stop the sideways direction movement 404 and to allow substantially only a movement in the longitudinal direction 403 by means of a wheel motion of wheel body 302. In this case, the roller braking device is activated while the wheel braking device is not activated.

FIG. 6 shows the omni wheel arrangement of FIG. 4 with a locked wheel movement according to an exemplary embodiment of the present invention. As indicated by symbols 601, the wheel motion of wheel body 302 is blocked. Rollers 303 are released and can freely move in the sideways direction 404. The arrangement of FIG. 6 with locked longitudinal movement allows for a wig-wag motion 602 of the whole mobile surgery system mounted on platform 409. Therefore, locking the wheel by means of wheel braking device and releasing or unlocking rollers 303 can enable a pure wig-wag motion 602. In this case, the wheel braking device is activated while the roller braking device is not activated.

FIG. 7 shows the omni wheel arrangement of FIG. 4 where wheels and rollers are blocked according to an exemplary embodiment of the present invention. Indicated in FIG. 7 is blocked wheel 601 and blocked rollers 501 resulting in a braking status where motion of platform 409 is prevented. If all wheels and all rollers 303, 302 are blocked, then all movements are arrested or locked.

Wheels with individual locks and/or individual brakes for rollers 303 and wheels 302 can be used to design out and omit the longitudinal and wig-wag mechanism inside mobile C-arms. The individual brakes are separate from another and may be controlled or activated independently from another.

In other words, the function of the longitudinal and the wig-wag mechanism is transferred from a complex mechanical move system into an omni wheel with accordingly lockable and/or releasable rollers and/or wheels. With this braking mechanism, low cost and un-motorized manual systems can be provided. Reducing the size of a longitudinal and wig-wag mechanism can lead to a compact and lightweight system which reduces the space consumption when storing such a system and when such a system is used.

FIG. 8 shows an omni wheel arrangement with two parallel omni wheels mounted in a front part of a platform according to an exemplary embodiment of the present invention. In FIG. 4, the omni wheel 312′ is mounted on the longitudinal axis 410 of the triangular-shaped base plate 409. According to the configuration of FIG. 8, two omni wheels 312′ are mounted symmetrically and parallel to the longitudinal axis 410. The double omni wheel configuration of FIG. 8 allows for carrying a heavy medical system mounted on the plate 409. The weight of the load is substantially equally distributed to both omni wheels.

For an omni wheel, the forward rolling diameter is used for substantially all longitudinal movements. This forward rolling diameter is formed by the wheel diameter. This forward rolling diameter is larger than the diameter of the rollers used for the sideways movement. For example, the wheel diameter of wheel 302 is 120 mm and the roller diameter is 25 mm. Smaller diameters of wheels result in high rolling friction and offer high resistance by climbing over floor irregularities or cables lying on the floor. Thus, according to FIGS. 1 to 8, the orientation of omni wheels 312′ is such that a forward rolling omni wheel is used. For sideways movements rollers are used. In some cases, a cross/sideways wheel configuration may be used, where the wheel 302 is oriented in the sideways direction.

FIG. 9 shows a double omni wheel sideways rolling configuration according to an exemplary embodiment of the present invention. Single and multiple sideways rolling omni wheel configuration where the wheel body 302 is directed to the sideways direction may be used if a large rolling diameter is necessary for the sideways movement. The principles as described in FIG. 1 to FIG. 8 also apply for the double sideway rolling omni wheel.

The configurations shown in FIGS. 1 to 9 use the locking and/or braking mechanisms on corresponding wheels and rollers on all omni wheels in the same way. A braking device or locking device for a wheel may be implemented on the main omni wheel shaft 301.

As an alternative to an electromagnetic brake the braking device or locking device for a roller can be implemented by pressing a plate on the side of the omni wheel as indicated with plates 307a, 307b, 307c, 307d.

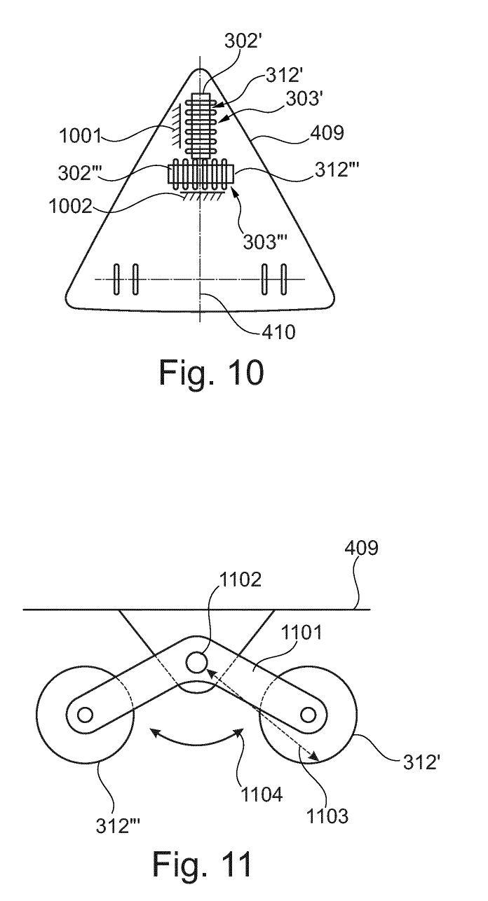

FIG. 10 shows an omni wheel arrangement using a combination of forward and sideways oriented omni wheels according to an exemplary embodiment of the present invention. Omni wheels 312′, 312′″ are mounted in a T-shape configuration along the longitudinal axis. The T-shape is oriented symmetrically to longitudinal axis 410 pointing into the direction of a forward movement. Thus, the T-shaped arrangement results in the omni wheel 312′, which is used for a longitudinal wheel movement, being mounted on the symmetry axis 410 or longitudinal axis 410 and the omni wheel 312′″, which is used for a sideway wheel movement, being mounted perpendicular to the longitudinal axis 410. As indicated with symbols 1001 and 1002, only a wheel part of the omni wheels 312′, 312′″ is locked in order to guarantee the corresponding movement. Braking devices for rollers 303′, 303′″ are not used in the configuration of FIG. 10. The two omni wheels 312′, 312′″ are positioned one behind the other. Each of the omni wheels is lockable on the wheel in order to allow for the desired movement. For example, for a sideways movement, wheel 312′ is blocked and for a longitudinal movement, wheel 312′″ is blocked. In particular, corresponding wheels 302′, 302′″ or wheel bodies 302′, 302′″ are blocked by the braking devices. The rollers 303′, 303′″ are not blocked since blocking of the wheel may be easier than blocking of the rollers. In another configuration the wheel is unblocked but rollers may be blocked if present. However, in order to reduce complexity blocking of rollers may not be provided.

FIG. 11 shows a pivot mounting configuration of two omni wheels according to an exemplary embodiment of the present invention. Even if by using the round shape for both wheels 312′, 312′″ in FIG. 11 a configuration with two similarly oriented wheels is indicated it is to be noted that the wheel configuration of FIG. 11 may also be the configuration as shown in FIG. 10, i.e. T-shaped. In that case one straight 312′ and one perpendicular oriented 312′″ omni wheel is used. Omni wheels 312′, 312″ can be mounted on a wing 1101 which has a pivot mounting 1102 to the plate 409. Mounting on a pivot can be used for both, the parallel wheel mounting and the T-shaped wheel mounting and in both cases help sharing the load.

In the following description it is assumed that the trailing wheel 312′″ is perpendicular to the leading wheel 312′. By using pivot 1101 and pivot joint 1102, the load from the plate 409 is equally distributed to the leading omni wheel 312′ and the trailing omni wheel 312′″. This equal load balancing helps for an equal distribution of braking traction in both directions. The virtual diameter as indicated by arrow 1103. The virtual diameter 1103 is larger than the individual wheel diameter of wheels 312′, 312′″. With such a configuration, climbing over small steps, floor variations and/or cables is easy due to the rocking action of a pivot 1102 as indicated by arrow 1104.

The single wheel configuration of FIG. 4 may work for small loads. So this solution with one single omni wheel may be used for light systems. The configuration with the wheel portion of omni wheel directed into the sideways direction may rarely be used. The configuration with the double omni wheel directed in the forward direction according to FIG. 8 with laterally spaced rollers may only be used for complex designs since the roller is an over constraint for wig-wag. If a double wheel configuration as of FIG. 8 is used and a wig-wag movement is desired it is to be considered that an instantaneous motion direction of wheels 312′ lies on a circle. However, in the double wheel configuration of FIG. 8 the orientations of wheels 312′ differ from the orientation of a radial direction directed from the center of the wig-wag motion to the center of the corresponding wheel. The center of the wig-wag motion is assumed to be in the middle of wheels 401′, 401″. Thus, the actual instantaneous motion direction of wheels 312′ when making a wig-wag movement is in a tangential direction of that circle. Therefore, the instantaneous movement may slightly differ from the circle direction. Consequently, it may be necessary for the wheels in order to follow the wig-wag movement to slightly slip. In order to prevent an uncontrollable slip that may result in a high rolling resistance and an inaccurate motion a configuration as of FIG. 10 may be employed.

The configuration according to FIG. 9 where wheel portion 302 of a double omni wheel is directed sideways may exhibit a high resistance for a forward motion due to the small roller radius directed in the forward direction.

The T-shape arrangement of FIG. 10 provides a broad range of application scenarios. This configuration according to FIG. 10 with omni wheels mounted in perpendicular directions is a good solution with regard to load bearing capacity and movability into all directions.

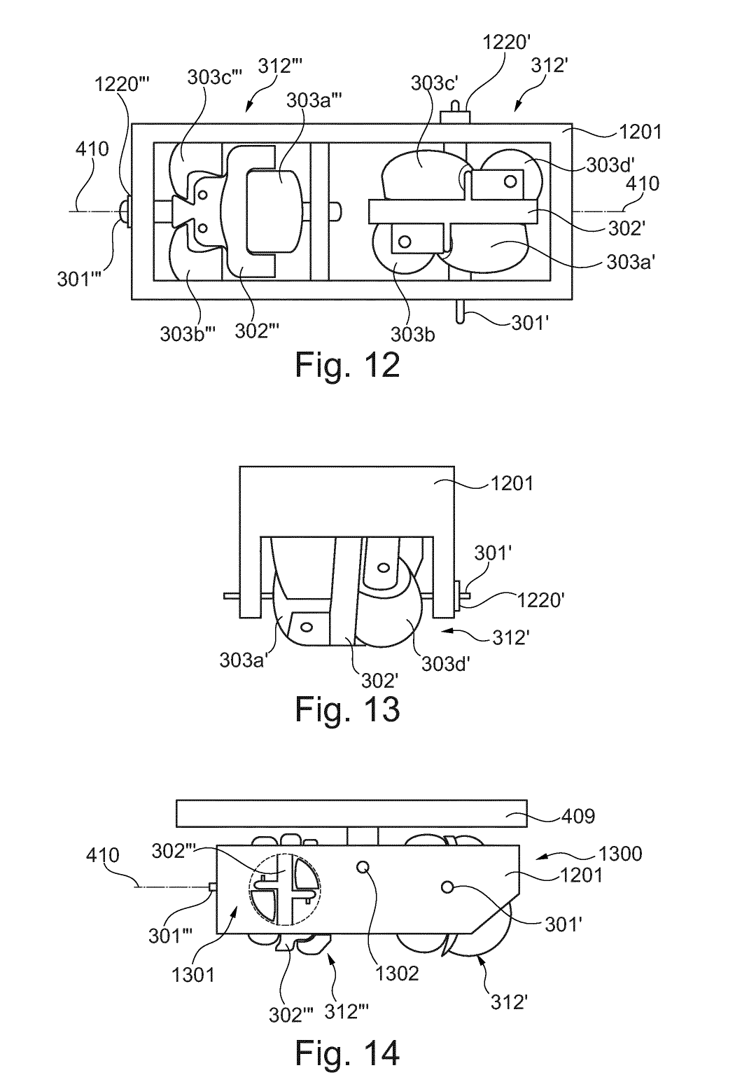

FIG. 12 shows a bottom view of a T-shape omni wheel arrangement for a configuration according to FIG. 10 according to an exemplary embodiment of the present invention. Housing 1201 is used to mount the omni wheels 312′ and 312′″. Longitudinal axis 410 is parallel to wheel shaft 301′″ of wheel 312′″. Wheel body 302′″ is perpendicular to longitudinal axis 410. Rollers 303a′″, 303b′″ and 303c′″ are mounted in such a way that they support a movement in the direction of the longitudinal axis 410, e.g. a longitudinal movement.

The omni wheel 312′ is mounted on a wheel shaft 301′ which is perpendicular to the longitudinal axis 410. Rollers 303a′, 303b′, 303c′, 303d′ are mounted in such a way to support a movement perpendicular to the longitudinal axis 410 or a sideways movement. Wheel body 302′ is substantially directed in the direction of the longitudinal axis 410 in order to support the longitudinal movement. For controlling the movement of wheels 312′, 312′″ braking devices 1220′, 1220′″, such as electromagnetic brakes 1220′, 1220′″, are mounted on corresponding wheel shafts 301′″. 301′. In order to reduce the complexity rollers 303a′, 303b′, 303c′, 303d′, 303a′″, 303b′″, 303c′″, 303d′″ have no brakes installed or are brakeless. Another example may also comprise rollers having brakes.

FIG. 13 is a front view of omni wheel assembly of FIG. 12 according to an exemplary embodiment of the present invention. FIG. 12 shows housing 1201 to which the omni wheel 312′ is mounted using wheel shaft 301′ or wheel axle 301′. Omni wheel 312′ comprises the wheel body 302′ and rollers 303a′, 303b′. As can be seen in FIG. 13, the circumference of wheel body in combination with the circumference of rollers can be used for contacting a surface. Both circumference surfaces supplement another to form a common circumference.

FIG. 14 shows a side view of the omni wheel arrangement of FIG. 12 according to an exemplary embodiment of the present invention. Omni wheel arrangement 1300 or omni wheel assembly 1300 is mounted on platform 409 of a medical device not shown in FIG. 14. The housing 1201 is used to mount omni wheels 312′ and 312′″ in a perpendicular orientation to another. The orientation or direction of an omni wheel may be substantially defined by the direction of the wheel device and/or of the wheel body. Wheel shaft 301′ is used to mount omni wheel 312′ and wheel shaft 301′″ is used to mount wheel 312′. The breakthrough view 1301 through the housing 1201 shows that wheel body 302′″ is arranged perpendicular to the longitudinal axis 410. Furthermore, pivot 1302 is shown which allows a pivot mounting of the omni wheel arrangement 1300 on platform 409 similar to the pivot mounting of FIG. 11.

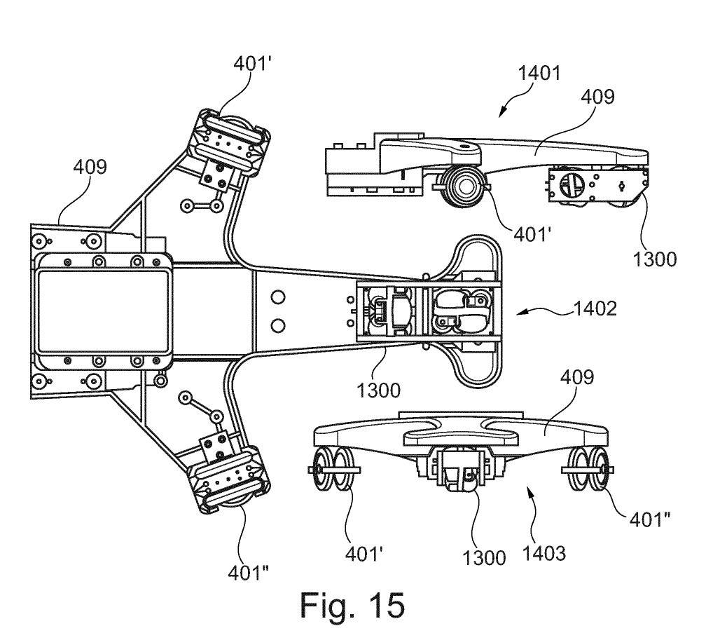

FIG. 15 shows a side view 1401, a bottom view 1402 and a front view 1403 of support plate 409 with an omni wheel arrangement according to an exemplary embodiment of the present invention. As can be seen in side view 1401 the support plate 409 and balancing wheels 401′ are combined with the omni wheel arrangement 1300. Bottom view 1402 shows the bottom side of base plate 409 to which balancing wheels 401′, 401″ and arrangement 1300 are mounted. The front view 1403 shows support plate 409, balancing wheels 401′, 401″ and omni wheel arrangement 1300.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfil the functions of several items re-cited in the claims. The mere fact that certain measures are re-cited in mutually different dependent claims does not indicate that a combination of these measures cannot be used to advantage. Any reference signs in the claims should not be construed as limiting the scope.

1. A mobile surgery system comprising at least one omni-wheel for moving the mobile surgery system, the omni-wheel comprising:
at least one wheel device having a wheel body;
a wheel shaft arranged in the center of the wheel body;
at least one roller device;
at least one of a wheel braking device and/or a roller braking device;
wherein the at least one roller device has a roller shaft and a roller body;
wherein the at least one roller device is arranged on the wheel body such that at least a part of the circumferential surface of the roller device forms at least a part of a circumferential surface of the wheel body;
wherein the wheel braking device is adapted for blocking a movement of the wheel body around the wheel shaft; and
wherein the roller braking device is adapted for blocking a movement of the roller body around the roller shaft, and
wherein the wheel braking device and the roller braking device are activatable independently.

2. The mobile surgery system of claim 1 wherein the at least one roller device is movable within the wheel device.

3. The mobile surgery system of claim 1 wherein a diameter of the roller device is smaller than the diameter of the wheel device.

4. The mobile surgery system of claim 3, wherein the at least one of a wheel braking device and a roller braking device comprises at least one of a braking plate, a motor and/or shaft blocking device.

5. The mobile surgery system of claim 4, further comprising:
a further omni-wheel;
a mounting plate;
wherein the at least one omni-wheel and the further omni-wheel are mounted on the same side of the mounting plate.

6. The mobile surgery system of claim 5, wherein the at least one omni wheel and the further omni-wheel are mounted on the mounting plate such that the shaft of the wheel-device of the at least one omni-wheel lies on the same virtual axis as the shaft of the wheel-device of the further omni-wheel.

7. The mobile surgery system of claim 5, wherein the at least one omni wheel and the further omni-wheel are mounted on the mounting plate such that the shaft of the wheel-device of the at least one omni-wheel lies on a virtual axis perpendicular to the virtual axis of the shaft of the wheel-device of the further omni-wheel.

8. The mobile surgery system of claim 5, wherein the at least one omni-wheel and the further -omni-wheel are mounted in a common housing.

9. The mobile surgery system of claim 5, wherein the at least one omni-wheel and the further -omni-wheel are mounted on a pivot.

10. An omni-wheel comprising:
a wheel device having a wheel body;
a wheel shaft arranged in the center of the wheel body;
at least one roller device;
at least one of a wheel braking device and a roller braking device;
wherein the at least one roller device has a roller shaft and a roller body;
wherein the at least one roller device is arranged on the wheel body such that at least a part of the circumferential surface of the roller device forms at least a part of a circumferential surface of the wheel body;
wherein the wheel braking device is adapted for blocking a movement of the wheel body around the wheel shaft; and
wherein the roller braking device is adapted for blocking a movement of the roller body around the roller shaft,
wherein the wheel braking device and the roller braking device are activatable independently.

11. An omni-wheel arrangement, comprising:
a first omni-wheel of claim 10;
a second omni-wheel of claim 10;
a mounting plate;
wherein the first omni-wheel and the second omni-wheel are mounted on the same side of the mounting plate.

12. A method for controlling an omni-wheel of claim 10, the method comprising:
determining a direction of desired movement;
if the direction of desired movement is parallel to the wheel shaft activating the wheel braking device;
if the direction of desired movement is perpendicular to the wheel shaft activating roller braking device;
if no movement is desired activating the wheel braking device and activating the roller braking device.

13. A method for controlling a mobile surgery system of claim 7, the method comprising:
determining a direction of desired movement;
if the direction of desired movement is parallel to the wheel shaft of the at least one omni wheel activating the wheel braking device of the wheel of the omni wheel and unlocking the wheel braking device of the further omni wheel perpendicular to the at least one omni wheel;
if the direction of desired movement is perpendicular to the wheel shaft of the at least one omni wheel activating the roller braking device of the at least one omni wheel and/or activating the wheel braking device of the further omni wheel;
if no movement is desired activating the wheel braking device of at least one omni wheel and/or the wheel braking device of the further omni wheel.

14. Program element for controlling an omni-wheel and/or a mobile surgery system, which, when being executed by a processor is adapted to carry out at least one of the method of claim 12.

15. Computer readable medium for controlling an omni-wheel and/or a mobile surgery system, comprising program code, which, when being executed by a processor is adapted to carry out at least one of the method of claim 12.
